(19) United States
(12) Patent Application Publication
(10) Pub. No.: US 2011/0212066 A1
(43) Pub. Date: Sep. 1, 2011

(54) METHOD OF FOSTERING THE RESTORATION OF A BODY FUNCTION USING CELLS OF THE ROOT SHEATH, COMPOSITION AND PREPARATION METHOD

(75) Inventor: Wolfgang RICHTER, Freising (DE)

(73) Assignee: EURODERM GMBH, Leipzig (DE)

(21) Appl. No.: 13/035,404

(22) Filed: Feb. 25, 2011

Related U.S. Application Data
(60) Provisional application No. 61/313,139, filed on Mar. 12, 2010.

(30) Foreign Application Priority Data
Feb. 26, 2010 (DE) 102010009571.0

(51) Int. Cl. A61K 35/36; C12N 5/07; C12N 5/077; C12N 5/079; A61P 17/00; A61P 25/00

(52) U.S. Cl. 424/93.7; 435/325; 435/378

(57) ABSTRACT

The present invention proposes a method for promoting the re-establishment of at least one function of the skin of a second skin area or of another area or part or tissue, comprising applying cells obtained from hair root sheaths of a first skin area of a donor onto a second skin area or another area or part or another tissue of a host. Furthermore, cells, a preparation, the use of cells and a method for preparing a preparation are proposed.

### CROSS-REFERENCE TO RELATED APPLICATIONS

The present application claims priority under 35 U.S.C. §119 of German Patent Application No. 10 2010 009 571.0, filed on Feb. 26, 2010 and claims the benefit under 35 U.S.C. §119(e) of U.S. Provisional Application No. 61/313,139, filed Mar. 12, 2010. The disclosures of these applications are expressly incorporated by reference herein in their entireties.

The present invention relates to a method for promoting the re-establishment of at least one function of the skin or of another tissue according to the preamble of claim 1. It further relates to cells according to claim 10, a preparation according to claim 15, the use of cells according to claim 16 as well as a method for producing a preparation according to claim 21.

The use of certain cells of the body at another body site than their original one is known from research literature. Thus, from PCT/EP 2008/007684 of the applicant of the present invention it is known that using melanocyte precursor cells of a first skin area increases the pigmentation of a second skin area.

One object according to the invention is to provide further uses of body cells.

This object of the present invention is achieved by a method according to claim 1. Additionally, cells according to claim 12, a preparation comprising cells according to claim 15, the use of cells according to claim 16 as well as a method for producing a preparation according to claim 19 are proposed.

According to claim 1, a method for promoting the establishment or re-establishment of one function of the skin and/or of another tissue is proposed. The method comprises applying cells obtained from hair root sheaths of a first skin area of a donor onto a second skin area of a host, another body part or another tissue of the recipient or host.

According to claim 10, cells of hair root sheaths of a first skin area of a donor are proposed that are or have been obtained for the purpose of being used in or on a second skin area, area or part or tissue for promoting the re-establishment of at least one function of the skin of the second skin area, of the area or part or tissue of a host.

According to claim 15, a preparation comprising the cells according to the invention is proposed.

According to claim 16, the use of cells that are or have been, respectively, obtained from hair root sheaths of a first skin area of a donor for preparing a preparation for promoting the re-establishment of at least one function of the skin of a second skin area, area or part or tissue of a host is proposed.

According to claim 21, a method for preparing a preparation, in particular a suspension, comprising cells of the hair root sheath is proposed.

Advantageous developments are subject-matter of the dependent claims and embodiments. In the following, the terms “may be” or “can be” or “may have” or “can have”, respectively, etc. shall be understood as synonyms for “preferably is” or “preferably has” etc. and shall illustrate one embodiment according to the present invention.

The method according to the invention of claim 1 in some embodiments according to the invention serves a non-therapeutic and non-surgical purpose.

Embodiments according to the present invention may comprise one or more of the following features.

If, in the following, reference is made to nerve cell precursor cells or stem cells this also applies without restriction to mesenchymal precursor cells even this is not explicitly stated. Nerve cell precursor cells and/or mesenchymal precursor cells are hereafter referred to as precursor cells; nerve cell stem cells and/or mesenchymal stem cells are referred to as stem cells.

The application of cells of the hair root sheaths, in particular of epithelial hair root sheaths, of a first skin area onto a second skin area serves to promote the re-establishment of at least one function of the skin of the second skin area, of a function of the second area or tissue or has this purpose.

As defined by the present invention, promoting the re-establishment of at least one function of the skin of a second skin area of a host in some embodiments according to the invention comprises promoting a re-innervation of the second skin area, of the area or part of the body or of the tissue. Thereby, in some embodiments, the application of the cells may advantageously contribute to inducing or stimulating a re-innervation of the skin or of (skin) cells. In certain embodiments of the method according to the invention, this contributes to or comprises an increased innervation in the context of promoting the re-establishment of at least one function of the skin of a second skin area or of another area or part or tissue.

In certain embodiments of the method according to the invention, the cells are nerve cell precursor cells and/or mesenchymal precursor cells (“precursor cells”) or comprise such cells. In some embodiments, said cells are present in a cell mixture that can be obtained when the cells that have been obtained from the hair root sheath are not divided with respect to their origin, their function or their character or nature. If, however, the cells are divided after they have been obtained, as defined by the present invention, a greater effect may be achieved after their application, when using primarily nerve cell precursor cells and/or mesenchymal precursor cells, particularly when using nerve cell precursor cells.

Applying the cells that have been obtained from hair root sheaths has the advantage of a substantially unlimited availability such that obtaining the cells without extracting skin, i. e., without any surgical invasion into the skin integrity at the extraction site, is advantageously possible. Due to the possibility of simply obtaining the cells of hair root sheaths in a high number, it may advantageously be possible to treat substantially unlimited skin areas. The application of cells from hair root sheaths thus implies their advantageously high availability.

In certain embodiments of the methods according to the invention, the cells that are used according to those methods are present in a suspension or in a sediment.

In certain embodiments of the present invention, the application of the cells is performed by simply brushing, spraying, dabbing, or the like. In some embodiments, the application is done non-invasively. In certain embodiments, it is done non-surgically. In some or all embodiments, application may be done without using physician or medical expertise.

Applying the cells may, e. g., be performed by using a suspension comprising 10²-10⁹ cells/ml, particularly 10⁵-10⁷ cells/ml, for example, by means of a syringe or a spray or in a biocompatible non-woven material.

The application can be done using a biocompatible solution (e. g., PBS) or by means of a biocompatible support (e. g., hyaluronan, collagen). Thereby, the cells may be used as vital cells or, e. g., as cells inhibited in their growth by using mitomycin C or radiation or as cell extracts (such as, e. g., lyophilisates, sonicates). Media conditioned with these cells can be used for this purpose as well.

The application of cells may be done by simply depositing the cells onto the skin. The cells may be fixed onto the second skin area by means of, e. g., a fibrin adhesive and may be secured by means of an occlusive or occlusion dressing. However, every other appropriate form of application, e. g., integrating the cells in biological or synthetic matrices is also possible according to the invention.

In one embodiment of the method according to the invention, the cells have been obtained from hair root sheaths of the first skin area by means of plucking hairs out of the vital skin of the donor or by means of plucking or otherwise obtaining hairs from or out of a present skin biopsy of the donor. Plucking the hairs in certain embodiments according to the present invention represents the only aspect of dividing and/or obtaining the cells from the first skin area. In those embodiments, it is thus exclusively plucked. It is particularly not cut, punched, or the like.

The cells can thus advantageously be obtained in a simple and thus repeatedly performable way. The process of obtaining the cells before their application may thus be performed in a comparably simple manner that is free of pain. Furthermore, this process does not include the risk of complications, in particular no or no significant risk of infection. In particular, no germs or pathogens are transferred when solely picking or plucking the hairs or their hair root sheaths whereas the such germs or pathogens are typically transferred in case of blood contact.

When using cells of hair root sheaths, the cells may, for example, be obtained by plucking scalp hair, in particular anagen hair, in particular hair of the capillitium, which as mentioned above has another advantage as compared to the use of cells of other origin, in particular of interfollicular stem cells.

“Obtaining” in the sense of the application in certain embodiments according to the invention refers to dividing or releasing the cells from the first skin area. According to the invention, the method may also comprise releasing the cells from the first skin area. This release may, for example, comprise a simple picking or plucking of the hairs, in particular anagen hairs of the scalp hair. In other embodiments according to the invention, obtaining explicitly does not include dividing the cells from the first skin area.

As defined by the application, in some embodiments, obtaining refers to dividing the cells, optionally the preparation as well, e. g., by means of trypsin; or comprises such a division and/or preparation.

According to the invention, besides dividing the cells from the first area—or alternatively hereto—, “obtaining” cells from hair root sheaths may comprise isolating the cells from the hair root sheaths, in particular from the epithelial hair root sheaths, as well.

The step of “obtaining” may also comprise one step or a plurality of steps by means of which the obtained cells are prepared for their application. This may be done by preparing a cell suspension (a cell suspension in general or a nerve cell precursor suspension and/or a suspension of mesenchymal precursor cells).

The cell suspension may in certain embodiments be prepared after an in vitro-propagation of the cells. In some embodiments according to the invention, cultivation is carried out.

However, the cell suspension may also be prepared directly, i e., without any further cultivation or growth cultivation of the cells for the purpose of cultivating, differentiating or maturating the cells therein.

In some embodiments of the method according to the invention, the cells are applied without having been cultivated in a growth cultivation.

In certain embodiments according to the invention, obtaining comprises releasing the cells from the skin or scalp regardless of the skin being vital skin or a skin biopsy.

Additionally to the cells and/or the precursor cells, the cell suspension may contain biocompatible substances such as PBS and/or a biocompatible support such as, for example, hyaluronan or collagen.

In a further embodiment of the method according to the invention, the second skin area, the second area or the second tissue is being prepared for receiving the cells. Such preparation allows for adhesion of the cells applied onto the second skin area, area or part or tissue in a particularly effective manner.

Preparing the second skin area may, for example, comprise releasing the epidermis or parts thereof from the second skin area. The latter is, for example, possible when using dermabrasio or superficial laser application accompanied by the advantages associated therewith and known to a person skilled in the art.

In certain embodiments according to the invention, preparation comprises applying an appropriate solution. An example of this is a fibrinogen solution preparing the second skin area for the subsequent reception of the cells and for a better adhesion of the cells and an, above all, cell growth at the second skin area.

In certain embodiments of the method according to the invention, the second skin area, area or tissue is not and/or not in the course of the method according to the invention is being prepared for receiving the cells. The latter, e. g., applies if the cells are applied onto an existing wound.

In some embodiments according to the invention of the method, the cells applied onto the second skin area, area or tissue are stimulated. In some embodiments, this, e. g., serves for an accelerated maturation or differentiation of the cells.

Such a stimulation may be carried out using UV radiation. The UV radiation activates the transferred cells and may, e. g., be done using broad band UV, narrow band UV, PUVA or excimer laser radiation.

Stimulating by using, for example, ultraviolet radiation may be done once or repeatedly. In doing so, radiation should advantageously be lower than the erythem limit. For example, radiating twice a week may result in a desired maturation of the second skin area. Radiating more or less frequently is also possible.

The applicant's studies have shown that stimulation by means of radiation can also further promote wound healing. This was shown after one UV stimulation.

The cells can be derived from one donor and can be re-applied to the donor again.

In some embodiments according to the invention, donor and host are identical. The first skin area and the second skin area are thus skin areas of one and the same individual. In such a case, efforts relating to typing or matching due to genetic differences between donor and host may advantageously be omitted or reduced. Additionally, the risk of transferring—for example, infections—from the donor to the host may be eliminated.

However, the cells can also be derived from a donor and can be applied to a host other than the donor. Thereby, it is irrelevant if donor and host are human or animal. A transfer between animal and human or vice versa is encompassed by the invention as well.

According to the invention, “cells” or “precursor cells”, respectively, are to be understood as both autologous and allogenic and xenogenic cells or precursor cells/stem cells, respectively.

The object according to the invention is further achieved by means of cells according to the features of claim 10. Advantageous developments are hereby in turn subject-matter of respective sub-claims.

As the same advantages that can be achieved by means of the method according to the invention described above may be achieved by means of the cells according to the invention, reference is made to the discussion above in order to avoid repetitions.

The cells according to the invention have been obtained from hair root sheaths of a first skin area. The cells according to the invention are thus present outside the body.

The cells according to the invention are, in some embodiments according to the invention, suited and provided for use in or on a second skin area, an area or a tissue for re-establishing at least one function of the skin of the second skin area.

In certain embodiments, the cells according to the invention have been obtained by means of plucking hairs out of the vital skin of a donor or by means of plucking or otherwise obtaining hairs from or out of a present skin biopsy of the donor.

In some embodiments, the cells have been obtained and are provided for their use without being or having been cultivated in a cell growth cultivation.

In certain embodiments, the cells are not or will not be changed or altered, in particularly not genetically altered.

The object according to the invention is further achieved by means of a preparation having the features of claim 15.

As the same advantages that can be achieved by means of the method according to the invention described above may be achieved by means of the preparation according to the invention, reference is made to the discussion above in order to avoid repetitions.

The preparation according to the invention comprises cells according to the invention.

Furthermore, the object according to the invention is also achieved by means of a method having the features of claim 16 or of claim 21. The advantages resulting from this method are the same advantages that result from the method described above to which reference is made in order to avoid repetitions.

Thereby, the cells could have been obtained by means of plucking hairs out of the vital skin of the donor or by means of plucking or otherwise obtaining hairs from or out of a present skin biopsy of the donor.

In one embodiment of the method according to the invention, the cells have been obtained and are provided for use without being or having been cultivated in a cell growth cultivation.

According to claim 21, a method for preparing a preparation is proposed. In some embodiments thereof, the preparation is a suspension. In certain embodiments, the preparation is provided and suited for use in any one of the methods described herein-above.

The method according to the invention for preparing a preparation, in particular a suspension, in some embodiments comprises at least enzymatically detaching the cells from the hair root sheath of an extracted hair. The enzymatic detachment may, for example, be done using a trypsin/EDTA solution.

EDTA may be present as a liquid in form of a clear, colorless, odorless solution, prepared according to Ph. Eur. (European pharmacopoeia in the current edition), having a pH of 5.42 to 6.04 and an osmolarity of 331-368 mOsm/kg such as is, for example, available from the company Biochrom AG, Germany under the article number L2113 in a 100 ml-glass bottle.

The trypsin/EDTA solution can be 0.8%. The solution particularly effects the detachment of epithelial cells from the hair sheath. The detachment is preferably carried out at 37° C. However, higher temperatures, at which the viability of the cells can still be ensured, or lower temperatures, at which the activity of trypsin can still be ensured, are possible as well.

For the enzymatic detachment, an incubation in trypsin, 0.1% to 10%, in particular between 0.5% and 4%, in PBS for 1-50, in particular for 15-30 min can be carried out.

PBS may be obtained by the company BioConcept, Switzerland, in a 500 ml-flask in liquid form having a pH of 7.3±0.2 and an osmolarity of 285±10 in form of PBS without Ca/Mg having the article number 3-05F29 (PBS1) or in form of PBS comprising Ca/Mg having the article number 8-05F00 (PBS2) as a sterile, colorless and clear liquid that can be stored at room temperature. Such PBS may be suited for preparing an M solution.

The particular advantage of the enzymatic detachment is that, due to the possible use of enzymes, an adverse effect on the cells treated and/or the change or alteration thereof, i. a. a genetic change, can be avoided. Thus, enzymatic detachment is particularly gentle for the cells to be obtained.

Additionally, the method for preparing the suspension in some embodiments according to the invention—alternatively or additionally and independently from each other, respectively—comprises the following steps: a) stopping the enzymatic detachment by, e. g., adding human serum; b) centrifuging the suspension in order to obtain a sediment, c) re-suspending the cellulous sediment in a thrombin solution allowing for an immediate fixation of the cells applied in a thin layer for their application on fibrinogen that has been applied previously and thus enabling a homogenous not too occlusive application in every body region.

As the adhesive protein solution, TissueCol-DuoS 2 ml Immuno can be used that is, for example prepared by the company Baxter AG, Austria under the article number B1332020110614 and available by the company Baxter Deutschland GmbH, Hyland Immuno Division, Germany. Such a biological two-component adhesive consists of 2 ready-to-use syringes comprising 2 ml adhesive protein solution with fibrinogen and 2 ml thrombin solution, respectively. After mixing the two components, solidification or hardening of the adhesive may be effected in seconds to minutes.

In some embodiments, the method comprises—independently from each other, respectively—the following steps: transferring the cells or the suspension or the sediment into a biocompatible solution, introducing or inserting the cells or the suspension or the sediment into a biocompatible support and/or preparing a cell extract.

In the following, exemplary embodiments are specified in detail.

Thus, in a first exemplary embodiment, a preparation was prepared. For this, different solutions have been prepared that would theoretically be sufficient for four patients and that can variably be adapted for each individual case.

A dispase solution has been prepared from 20 ml dispase and 40 ml PBS 1 and has been resuspended in 250 ml-culture medium flasks. Thereafter, 4 aliquots of 15 ml each have been transferred into 50 ml-tubes for hair plucking.

PBS 1 has been aliquoted in 4×8 ml each for transferring the hair follicles into 50 ml-tubes.

For the trypsin solution, 4 ml trypsin solution (e. g., TS solution from Sigma), 2 ml EDTA (1.0% from Biochrom) and 4 ml PBS1 were mixed and aliquoted in 15 ml-tubes with 2 ml each.

The stopping solution has been prepared by mixing 135 ml PBS2 with 15 ml human serum and resuspending the mixture in 250 ml-culture medium flasks, Thereafter, the solution was aliquoted in 50 ml-tubes with 30 ml each.

For the thrombin solution, 2 ml thrombin (TissueCol-DuoS) were mixed with 11.3 ml PBS2 (with Ca/Mg) and resuspended in 15 ml-tubes. For transportation, 2 ml were drawn into 2 ml-syringes comprising a sterile cannula (size 1) each, the air bubbles were removed, the cannula was provided with a protective cover again, the syringes were packed in adhesive bags and were sent in a non-frozen state together with a thermal pack.

The fibrinogen solution was prepared by mixing 2 ml fibrinogen (TissueCol-DuoS) with 6 ml PBS1. For transportation, 2 ml were drawn into 2 ml-syringes comprising a sterile cannula (size 1) each, the air bubbles were removed, the cannula was provided with a protective cover again, the syringes were packed in adhesive bags and were sent in a non-frozen state together with dry ice.

Per patient and/or treatment, the following devices or apparatuses and/or consumables or expandables, respectively, may be used or optionally be sent together with a treatment set or kit or be prepared or provided, respectively, for the treatment: pipettor and charging cable, sterile metal forceps, a 90 ml-petri dish, pipettes (10 pipettes with 10 ml/5 pipettes with 2 ml), a cellular sieve, 1-2 cryo-tubes; a 50 ml-tube containing 15 ml dispase solution, a 50 ml-tube containing 8 ml PBS1, a 15 ml-tube containing 2 ml trypsin solution, a 50 ml-tube 30 ml PBS2/human serum, a 50 ml-tube (for the cellular sieve), four 15 ml-tubes (for centrifugation); a syringe (comprising a cannula) containing 2 ml fibrinogen solution, a syringe (comprising a cannula) containing 2 ml thrombin solution.

At first, a cell suspension was prepared. For that purpose, a fibrinogen solution (syringe) was thawed at room temperature. A dispase solution for hair plucking (15 ml) was transferred into a 90 ml-petri dish. Thereafter, the main part of the hair (dead hair material) was cut from about 250 hair follicles (may be sufficient for treating an area of about 20 to 30 cm²) that are already present in a picked state. The hair follicles were transferred into the 90 ml-petri dish.

The cut hair follicles were transferred into a 50 ml-tube containing 8 ml PBS 1 by means of a sterile forceps. Then, 2 ml trypsin solution was added. Attention should be paid that all hair follicles are immersed into the solution.

The tube was heated (e. g., by means of a thermal block/water bath) to about 37° C. The trypsin treatment was performed for about 25-30 min under occasional shaking/resuspending. By adding 30 ml PBS2/serum, trypsin was deactivated.

A mechanical detachment of the nerve cell precursor cells was obtained by thoroughly pipetting (at least 30 times), e. g., by means of a 10 ml-pipette.

Then, the suspension was transferred through a cellular sieve (pore size 70 μm) into a 50 ml-tube in order to remove dead cell material/cell aggregates.

The suspension (40 ml) was distributed to four 15 ml-tubes. The cells were centrifuged and the supernatant was poured or pipetted away. The thrombin solution (2 ml) was added from the syringe to the cell pellet and the cells were resuspended by using a 2 ml-pipette. Thereby, the cells of the four tubes were collected.

It is indicated that, in some embodiments of the method according to the invention, centrifugation is not provided. In those embodiments, the method can be performed without using centrifugation. This may advantageously minimize the technical effort required for performing the method.

Subsequently, the cell suspension was transferred into a cryo-tube and the drawn into the thrombin syringe.

For treating superficial skin wounds that have been generated by means of abrasion, 2 ml fibrinogen solution was applied onto the patients' wound area. Then, the thrombin cell suspension was applied. The wound was occluded in a common way. The dressing was changed after 5 days.

Likewise, for treating scar tissue i.a. due to the lack of sensation of the scar (the same is possible for keloid formation), a pre-treatment of the patients was done in the following way. After local anesthesia of the wound, the scar tissue was ablated using dermabrasio or an erbium YAG laser. Then, the method according to the invention started with applying the cell suspension and the fibrin adhesive (optionally) by means of a syringe or a spraying device. The areas treated were covered with a plastic dressing. The dressing was changed after 5 to 7 days.

In the following, the treatment of a patient for which re-establishment of sensation of the patient within the area of the second skin area was observed will be described exemplarily:

The patient, male, 29 years old had a scar on the upper part of the body resulting from an operation dating back several years. This has been treated after a skin abrasion by means of the method according to the invention in the following way. Thereby, nerve cell precursor cells were applied to the patient.

After a single treatment using the method according to the invention in 2009, the acute wound that had been generated by means of dermabrasio prior to applying the method according to the invention occluded. Thereby, the patient observed a re-establishment of feeling or sensation in the newly formed scar. Therein, the scar that formed after applying the method according to the invention differed from the scar that had formed after the original operation on the upper part of the body. The latter one had healed without establishing any sensation across the scar area. The sensation had not returned several years after the operation. However, by means of the method according to the invention, the sensation and the ability of the patient to discriminating stimuli on the newly formed scar tissue have been re-established.

1.-24. (canceled)

25. A method of promoting a re-establishment of at least one function of skin of a skin area or of another area or part or tissue, wherein the method comprises applying cells obtained from hair root sheaths of a first skin area of a donor onto a second skin area or onto another area or part or onto another tissue of a host.

26. The method of claim 25, wherein the cells are or comprise at least one type of precursor cells selected from of nerve cell precursor cells and mesenchymal precursor cells.

27. The method of claim 25, wherein promoting the re-establishment of at least one function of the skin of a skin area or of another area or part or tissue is or comprises an increased innervation.

28. The method of claim 25, wherein the cells have been obtained from hair root sheaths of the first skin area by means of plucking hair out of vital skin or out of a skin biopsy of the donor or have been obtained otherwise from or out of a skin biopsy of the donor.

29. The method of claim 25, wherein the method further comprises preparing the obtained cells before an application thereof.

30. The method of claim 25, wherein the method further comprises applying the obtained cells without using any prior cell growth cultivation.

31. The method of claim 25, wherein the method further comprises preparing the second skin area, area or part or tissue for receiving the obtained cells.

32. The method of claim 25, wherein the method further comprises stimulating the cells after application onto the second skin area, area or part or tissue.

33. The method of claim 25, wherein donor and host are identical.

34. A plurality of cells from hair root sheaths of a first skin area of a donor, wherein the cells are used in or on a second skin area, another area or part or another tissue of a host for promoting a re-establishment of at least one function of the skin of the second skin area, the other area or part or the other tissue of the host.

35. The plurality of cells of claim 34, wherein the cells are or comprise precursor cells selected from at least one of nerve cell precursor cells and mesenchymal precursor cells.

36. The plurality of cells of claim 34, wherein promoting a re-establishment of at least one function of skin of a skin area or of another area or part or tissue is or comprises an increased innervation.

37. The plurality of cells of claim 34, wherein the cells have been obtained by plucking hairs out of vital skin or a skin biopsy of the donor or otherwise from or out of a skin biopsy of the donor.

38. The plurality of cells of claim 34, wherein the cells are obtained and provided or intended for being applied onto a second skin area, area or part or tissue of a host or for preparing a preparation without being cultivated or having been cultivated in a cell growth cultivation prior to their application or preparation.

39. A preparation which comprises the plurality of cells of claim 34.

40. A method of preparing a preparation comprising cells from hair root sheaths, wherein the method comprises one or more of the following operations:
(i) enzymatically detaching cells from a hair root sheath;
(ii) stopping the enzymatic detachment of (i);
(iii) centrifuging or filtering a cell suspension for obtaining a sediment or a cell concentrate;
(iv) suspending the cell sediment or cell concentrate of (iii);
(v) transferring the cells of (i) or (ii) or the suspension or the sediment or the concentrate of (iii) into a biocompatible solution;
(vi) inserting or introducing the cells of (i) or (ii) or the suspension or the sediment or the concentrate of (iii) into a biocompatible support;
(vii) preparing a cell extract.

41. The method of claim 40, wherein the preparation is a suspension.

42. The method of claim 40, wherein the cells comprise one or more of nerve cell precursor cells and mesenchymal precursor cells.

43. The method of claim 40, wherein (ii) comprises adding human serum.

44. The method of claim 40, wherein (iv) comprises suspending the cell sediment or cell concentrate of (iii) in a thrombin solution.

45. The method of claim 40, wherein the method does not comprise cultivating the cells from the hair root sheath.

46. The method of claim 40, wherein the method does not comprise (ii).

47. The method of claim 40, wherein the method does not comprise (iii).
